(19) United States
(12) Patent Application Publication
(10) Pub. No.: US 2026/0310233 A1
(43) Pub. Date: Oct. 8, 2026

(54) INTELLIGENT POSITIONING OF ROBOT ARM CART

(71) Applicant: Mazor Robotics Ltd., Caesarea (IL)

(72) Inventors: Weijun Xu, Shanghai (CN); Wei Tang, Shanghai (CN); Fang Geng, Shanghai (CN)

(21) Appl. No.: 19/482,380

(22) PCT Filed: May 8, 2023
(86) PCT No.: PCT/CN2023/092729

§ 371 Date: Nov. 7, 2025

(51) Int. Cl. G05D 1/689; A61B 34/20; A61B 34/32; G05D 1/622; G05D 107/60; G06T 7/73

(52) U.S. Cl. CPC G05D 1/6895; A61B 34/20; A61B 34/32; G05D 1/622; G06T 7/74; G05D 2107/65; G06T 2207/10016; G06T 2207/10048; G06T 2207/30204

(57) ABSTRACT

A surgical system, navigation system, and method are provided. An illustrative system includes a robot mounted to a movable base, the robot including one or more robotic arms, a processor, and memory coupled with the processor. The memory includes data stored thereon that, when processed by the processor, enables the processor to: receive image data describing a position of the one or more robotic arms relative to a surgical target area; determine a current position of the robot is sub-optimal for enabling the robot to access the surgical target area; and provide at least one of instructions and animations for moving the movable base from the current position to a new position.

### FIELD

The present disclosure is generally directed to surgical systems, and relates more particularly to robotic surgical devices.

### BACKGROUND

Surgical robots may assist a surgeon or other medical provider in carrying out a surgical procedure, or may complete one or more surgical procedures autonomously. Layout of the operating room during a surgical procedure is especially important to support successful use of surgical robots.

### BRIEF SUMMARY

Example aspects of the present disclosure include:

A system including: a robot mounted to a movable base, the robot comprising one or more robotic arms; a processor; and memory coupled with the processor, the memory comprising data stored thereon that, when processed by the processor, enables the processor to: receive image data describing a position of the one or more robotic arms relative to a surgical target area; determine a current position of the robot is sub-optimal for enabling the robot to access the surgical target area; and provide at least one of instructions and animations for moving the movable base from the current position to a new position.

In some aspects, the instructions cause the movable base to autonomously move from the current position to the new position.

In some aspects, the data, when processed by the processor, further enables the processor to: determine a proposed path from the current position to the new position.

In some aspects, the proposed path avoids at least one obstacle and remains in a sterile area.

In some aspects, the image data comprises an image of one or more tracking objects positioned in proximity with the surgical target area.

In some aspects, the one or more tracking objects are mounted to at least one of an end effector and the one or more robotic arms.

In some aspects, the one or more tracking objects are mounted to a patient anatomy.

In some aspects, the one or more tracking objects are mounted to a surgical instrument.

In some aspects, the image data is obtained from an imaging device and wherein the imaging device comprises at least one of an infrared imaging device, an optical imaging device, and a video camera.

In some aspects, the instructions are provided for moving the movable base from the current position to an new position and wherein the instructions include an indication of whether or not the movable base is located in the new position.

A navigation system is also provided that includes: an imaging device; a processor; and memory coupled with the processor, the memory comprising data stored thereon that, when processed by the processor, enables the processor to: receive image data from the imaging device, wherein the image data comprises an image of a robotic arm and an image of a surgical target area; determine a position of the robotic arm; determine a position of the surgical target area; determine, based on the position of the robotic arm and the position of the surgical target area, that a cart supporting the robotic arm is sub-optimally placed relative to the surgical target area; and provide at least one of instructions and animations for moving the cart from a current position to a new position.

In some aspects, a tracking object is mounted on or held by the robotic arm, wherein the image data includes an image of the tracking object, and wherein the position of the robotic arm is determined by analyzing the image of the tracking object.

In some aspects, the data, when processed by the processor, further enables the processor to: determine a proposed path from the current position to the new position; and display the proposed path via a user interface.

In some aspects, the data, when processed by the processor, further enables the processor to: determine an obstacle is precluding the robotic arm from achieving a desired pose to enable an end effector to access the surgical target area; and determine that the new position enables the end effector to access the surgical target area.

In some aspects, the imaging device comprises at least one of an infrared imaging device, an optical imaging device, and a video camera.

A method is also provided that includes: receiving image data from an imaging device, wherein the image data comprises an image of a robotic arm and an image of a surgical target area; determining a position of the robotic arm relative to a position of the surgical target area; determining, based on the position of the robotic arm relative to the position of the surgical target area, that a cart supporting the robotic arm is sub-optimally placed relative to the surgical target area; and providing at least one of instructions and animations for moving the cart from a current position to a new position.

In some aspects, the method further includes outputting an indication when the cart is co-located with the new position.

In some aspects, the method further includes determining a path from the current position to the new position.

In some aspects, the method further includes causing the cart to move autonomously along the path.

In some aspects, the path avoids at least one obstacle and remains in a sterile area.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1 is a block diagram of a system according to at least one implementation of the present disclosure;

FIG. 2 illustrates additional details of a system according to at least one implementation of the present disclosure;

FIG. 3 is a plan view of an environment in which a surgical robot may operate according to at least one implementation of the present disclosure;

FIG. 4 is an example of a first process flow according to at least one implementation of the present disclosure;

FIG. 5 is an example of a second process flow according to at least one implementation of the present disclosure; and

FIG. 6 is an example of a third process flow according to at least one implementation of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,”“by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Some robotic surgical system implementations include operating a robotic system within proximity of a surgical table (e.g., a surgical bed, an operating table, etc.) and a patient. The term “robotic system” may also be referred to as a “robotic surgical system” herein.

In some robotic system implementations (e.g., in which the robotic system or a robotic arm is not table-mounted or patient-mounted), movement of the robotic system relative to the surgical table and/or patient during a surgical operation may become necessary or desirable. This can be achieved by moving the robotic system, which may be mounted on a cart or other moveable device. This can alternatively or additionally be achieved by moving the surgical table.

Aspects of the present disclosure support improving positioning of the robotic system relative to the surgical table, the patient, or a surgical target area.

In many traditional robotic systems, there is no guidance for the robot arm cart positioning. The surgeon assumes a positioning of the robotic system and it's components (e.g., arm, end effector, etc.) are correct for the surgical procedure. This assumption can be flawed or conditions may change during the surgical procedure that result in the robotic system not having full access to the surgical target area.

Repositioning the robot arm cart, especially during a surgical procedure, can waste important time or unnecessarily extend the time of the surgical procedure.

One aspect of the present disclosure is to provide an approach for determining whether or not a robot and components thereof are in an appropriate or optimal position relative to a surgical target area. Such determinations may be made during initial setup of the operating room or during the surgical procedure. Aspect of the present disclosure also provide suggestions for improving a position of the robot relative to the surgical target area. In some examples, the robot may be mounted on a cart (e.g., a robot arm cart), and suggestions, instructions, and/or animations for moving the cart can be provided to operating room personnel. In some examples, the cart may be provided with an ability to move autonomously or semi-autonomously, in which case the instructions for moving the cart can be provided directly to the cart, thereby enabling the cart to move according to a predetermined path within the operating room.

In some embodiments, navigation components (e.g., optical trackers or tracking objects) may be mounted at or near the surgical target area, on the robot end effector, on the robot arm, or combinations thereof. A navigation system may be provided with an ability to determine a position of the robot, the robot end effector, and the robot arm relative to the surgical target area by tracking a position of the navigation components. Based on determined relative positions of the robot, the robot end effector, the robot arm, and the surgical target area, the navigation system may also determine if the robot is optimally placed relative to the surgical target area, if an obstacle is precluding the robot from fully accessing the surgical target area, and/or if the robot should be moved. The system may also be capable of determining if the robot cart can be safely moved from its current position to an improved position (e.g., without impacting another obstacle, without impacting personnel, without leaving a sterile area, etc.). Similar approaches may be applied to non-robotic arms. For instance, approaches described herein may be used to determine whether a mechanical arm (different from a surgical robot arm) is able to move into a desired location to support a surgical procedure.

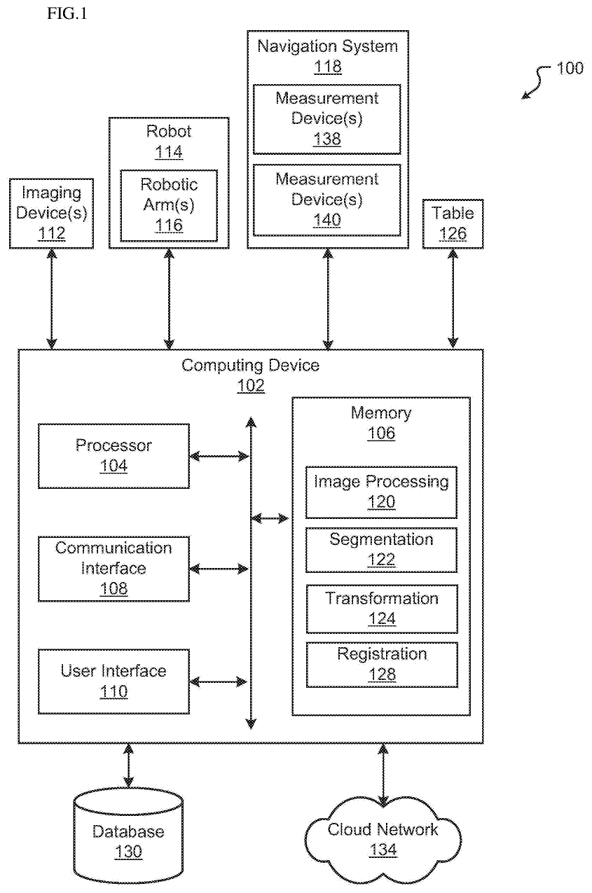

FIG. 1 illustrates an example of a system 100 that supports aspects of the present disclosure. The system 100 is illustrated to include a computing device 102, imaging devices 112, a robot 114, a navigation system 118, a table 126, a database 130, and/or a cloud network 134 (or other network). Systems according to other implementations of the present disclosure may include more or fewer components than the system 100. For example, the system 100 may omit and/or include additional instances of the computing device 102, imaging devices 112, the robot 114, the navigation system 118, measurement device 138, measurement device 140, the table 126, one or more components of the computing device 102, the database 130, and/or the cloud network 134. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry associated with controlling the imaging devices 112, the robot 114, the navigation system 118, and the table 126. The computing device 102 may also be, for example, a control device for autonomously or semi-autonomously controlling a cart on which the robot 114 is provided. The computing device 102 may also be, for example, a device which provides instructions, suggestions, and/or animations to operating room personnel (e.g., doctor, nurse, staff, etc.) for moving a cart on which the robot 114 is provided.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from imaging devices 112, the robot 114, the navigation system 118, the table 126, the database 130, and/or the cloud network 134. Alternatively or additionally, the processor 104 may include one or multiple processors.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the method 400 described herein, or of any other methods. The memory 106 may store, for example, instructions and/or machine learning models that support one or more functions the computing device 102, imaging devices 112, the robot 114, the navigation system 118, the table 126. For instance, the memory 106 may store content (e.g., instructions and/or machine learning models) that, when executed by the processor 104, enable image processing 120, segmentation 122, transformation 124, and/or registration 128). Such content, if provided as in instruction, may, in some implementations, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices 112, the robot 114, the navigation system 118, the database 130, and/or the cloud network 134.

The computing device 102 may also include a communication interface 108. The communication interface 108 may be used for receiving data or other information from an external source (e.g., the imaging devices 112, the robot 114, the navigation system 118, the database 130, the cloud network 134, and/or any other system or component separate from the system 100), and/or for transmitting instructions, data (e.g., image data provided by the imaging devices 112, measurement data provided by measurement device(s) 138, measurement device(s) 138, 140, etc. ), or other information to an external system or device (e.g., another computing device 102, the imaging devices 112, the robot 114, the navigation system 118, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto. As will be described in further detail herein, the user interface 110 may be used to display instructions and/or animations to operating room personnel regarding placement and/or positioning of the robot 114 relative to the table 126.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). The imaging device 112 may also be operable to capture discrete images or video images of an environment in which a surgical procedure is taking place. Illustratively, and without limitation, the imaging device 112 may be configured to capture images of a patient, of the table 126 on which the patient is located, objects surrounding the table 126, and tracking objects positioned within a field of view of the imaging device 112. Examples of tracking objects are further described in U.S. Pat. No. 9,179,984, assigned to Medtronic Navigation Inc., the entire contents of which are hereby incorporated herein by reference. Utilization of one or multiple tracking objects can be useful to produce image data that helps determine a location of a surgical target area and a position of the surgical target area relative to other devices on which tracking objects are placed (e.g., a robot 114, a robotic arm 116, an end effector of the robot 114, etc.).

“Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some implementations, the imaging device 112 may comprise more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure. The robot 114 may be configured to operate or control aspects of one or multiple measurement devices 138, 140.

The robot 114 may comprise one or more robotic arms 116. In some implementations, the robotic arm 116 may comprise a first robotic arm and a second robotic arm, though the robot 114 may comprise more than two robotic arms. In some implementations, one or more of the robotic arms 116 may be used to hold and/or maneuver the imaging device 112, a surgical instrument, or the like. In implementations where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more Degrees of Freedom (DoF). Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm). The robotic arm(s) 116 may include an end effector (not illustrated) coupled to a distal end of the robotic arm(s). The end effector may support interaction of the robotic arm(s) with an environment.

In some implementations, reference markers (e.g., navigation markers, three-dimensional markers, tracking objects, etc) may be placed on the robot 114 (including, e.g., on the robotic arm 116, on an end effector of the robot 114, etc.), the imaging device 112, the measurement device(s) 138, the measurement device(s) 140, the table 126, or any other object in the surgical space. The reference markers or tracking objects may be tracked by the navigation system 118, and the results of the tracking may be used by the robot 114 and/or by an operator of the system 100 or any component thereof. In some implementations, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, tracking objects, or other objects within the operating room or other room in which some or all of the system 100 is located. The one or more cameras may be optical cameras, RGB cameras, infrared cameras, or other cameras. In some implementations, the navigation system 118 may comprise one or more electromagnetic sensors. In various implementations, the navigation system 118 may be used to track a position and orientation (e.g., a pose) of the imaging device 112, the robot 114 and/or robotic arm 116, the measurement device(s) 138, the measurement device(s) 140, and/or one or more surgical tools (or, more particularly, to track a pose of a navigated tracker attached, directly or indirectly, in fixed relation to the one or more of the foregoing).

The system 100 may support alternative and/or additional implementations of coordinate measuring and coordinate tracking in association with the patient (e.g., an anatomical element of the patient, a surgical target area, or the like) using the measurement device(s) 138, 140, and/or image data. The navigation system 118 may include a display (e.g., display 242 later described herein) for displaying one or more images from an external source (e.g., the computing device 102, imaging device 112, or other source) or for displaying an image and/or video stream from the one or more cameras or other sensors of the navigation system 118. In some implementations, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, whether the robot 114 is positioned appropriately relative to a surgical target area, how to move the robot 114, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may additionally or alternatively store, for example, location or coordinates of objects (e.g., anatomical elements of a patient, the robot 114, the table 126, etc.) associated with the system 100. The database 130 may be configured to provide any such information to the computing device 102 or to any other device of the system 100 or external to the system 100, whether directly or via the cloud network 134.

In some implementations, the database 130 may include thresholds associated with movement of a patient, the robot 114, the measurement device(s) 138, the measurement device(s) 140, and/or the table 126. In some implementations, the database 130 may be or comprise part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/ or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1xRTT), evolution-data optimized (EVDO), high speed packet access (HSPA), universal mobile telecommunications service (UMTS), 3G, long term evolution (LTE), 4G, and/or 5G, etc. ), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means.

Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VOIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of the process flow 300 described herein. The system 100 or similar systems may also be used for other purposes.

Aspects of the system 100 supportive of monitoring patient movement are later described herein with reference to the following figures.

Referring to FIG. 2, examples of a system 200, which include a robotic system 201 are shown according example implementations of the present disclosure.

Features of the robotic system 201 may be described in conjunction with a coordinate system 202. The coordinate system 202, as shown in FIG. 2, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robotic system 201. These planes may be disposed orthogonal, or at 90 degrees, to one another. While the origin of the coordinate system 202 may be placed at any point on or near the components of the robotic system 201, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robotic system 201 with respect to the coordinate system 202.

FIG. 2 illustrates an example system 200 that supports aspects of the present disclosure. In the example, the robotic system 201 may include a robot 114 (e.g., electronic and mechanical components including robotic arm 216) mounted on or supported by a movable base 212. The movable base 212 for the robot 114 may also be referred to as a robot cart or robot arm cart. The system 200 further illustrates placement of the robot 114 and the moveable base 212 relative to a table 226. The table 226 may correspond to an example of table 126, which can also be referred to as a surgical table, an operating table, a patient bed, etc.

The robotic system 201 (e.g., robot 114, robotic arm 216, 220, etc.) may include examples of aspects of like elements described herein with reference to FIG. 1. In some cases, the robotic system 201 may be referred to as a workstation. For example, the robotic system 201 may include a display 242 and additional user interfaces (e.g., keyboard, mouse, controls, etc.) for manipulating the robot 114. Display 242 may correspond to an example of the user interface 110.

The robotic system 201 may include one or multiple robotic arms 216, 220, which may correspond to an example of robotic arm 116. In some embodiments, a tracking object 208 or optical navigation component may be secured onto or held by a robotic arm 216, 220. A robotic arm 216, 220 may be moved near a surgical target area, thereby providing a physical proximity between the tracking object 208 and the surgical target area of the patient 204. As shown in FIG. 2, the tracking object 208 may be attached to an end effector 224 of the robotic arm 220. It should be appreciated that the tracking object 208 may alternatively or additionally be attached to an intermediate arm or link (e.g., elbow). In some embodiments, the robotic system 201 may be configured to determine a distance (d1) between the patient 204 and the end of the table 226 and/or a distance (d2) between the patient 204 and the robot 114. The distances (d1 or d2) may be determined using sensors provided on the robot 114 or by using optical navigation as described herein. For instance, the navigation system 118 may be configured to determine a position of the tracking object 208 within the coordinate system 202, a position of the robot 114 within the coordinate system 202, and a pose of the robotic arms 216, 220 within the coordinate system 202. Such information can be determined using image data and may be useful in determining whether or not the robot 114 is at an ideal location relative to the table 226. The image data may also be useful in determining alternative or improved positions for the robot 114 and for suggestion such alternative or improved positions via the display 242.

Aspects of the robotic system 201 may support monitoring of patient movement (e.g., as provided by a measurement device 238 and/or by image data), monitoring of personnel movement (e.g., as provided by image data), monitoring of surgical target areas, and the like. In an example, the robotic system 201 may monitor patient movement (e.g., movement of an anatomical element) with respect to the robotic system 201. In some examples, the robotic system 201 may monitor patient movement with respect to the robot 114, the robotic arm 216, 220, and/or a surgical tool coupled to the robotic arm 216, 220. The robotic system 201, possibly with support of information from the navigation system 118, may also be configured to determine if an obstacle has moved between the robot 114 and the surgical target area, as will be described in further detail herein.

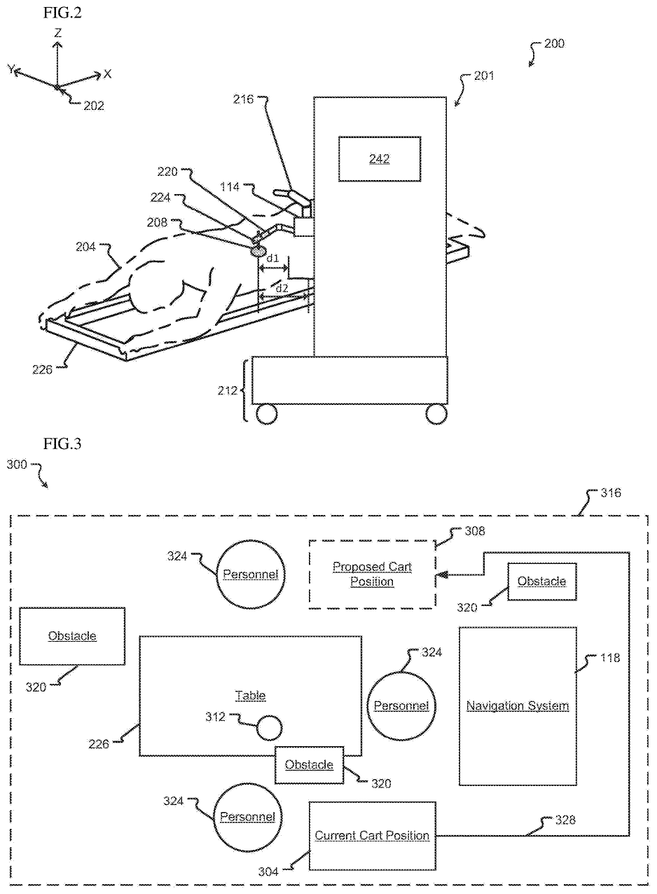

With reference now to FIG. 3, additional details of an environment 300 in which a robotic system 201 may operate will be described in accordance with at least some embodiments of the present disclosure. The environment 300 may include a surgical environment, such as an operating room. The environment 300 may include a sterile area 316 and a non-sterile area. Objects contained within the sterile area 316 may be considered sterile or “safe” as compared to objects located outside the sterile area 316.

In some embodiments, the table 226 and patient 204 may be provided within the sterile area 316 along with health care personnel 324 (e.g., doctors, surgeons, nurses, support staff, etc.). Some or all of the robot 114 may also be provided within the sterile area 316. As shown in FIG. 3, the robot 114 may initially be positioned at a first position (e.g., current cart position 304) relative to the table 226. The robot 114 may be utilized by personnel 324 during the surgical procedure to assist at or near the surgical target area 312.

During the surgical procedure, an obstacle 320 may move into a location that obstructs the robot's 114 access to the surgical target area 312. The obstacle 320 may partially or completely block the robot's 114 access to the surgical target area 312. In some examples, the obstacle 320 may block movement of the robotic arm 216, 220 to a preferred or desired position. In some examples, the obstacle 320 may preclude the end effector 224 from accessing the surgical target area 312. In situations where the current cart position 304 can be improved and the benefits associated with movement of the robot 114 outweigh potential risks associated with movement of the robot 114 (e.g., impacts, consumption of time, etc.), then an alternative or new proposed cart position 308 may be identified and suggested to personnel 324.

As shown in FIG. 3, a proposed cart path 328 may also be determined and suggested to personnel 324. The proposed cart path 328 may originate at the current cart position 304 and end at the proposed cart position 308. In some embodiments, the proposed cart path 328 may be required to remain within the sterile area 316 and may further be required to avoid obstacles 320 or objects within the environment 300. If a safe proposed cart path 328 cannot be achieved (e.g., due to potential impacts or due to requirements of remaining within the sterile area 316, then the proposed cart position 308 may be determined as unavailable or non-viable, in which case no new proposed cart positions 308 are provided to personnel 324. In other words, the system 100 may be enabled to determine: (1) if a current cart position 304 can be improved to access the surgical target area 312 and (2) if a viable cart path 328 is available to move the robot 114 from the current cart position 304 to the proposed cart position 308. If both conditions (1) and (2) cannot be satisfied, then the system 100 may determine the current cart position 304 is the best or optimal position for the robot 114.

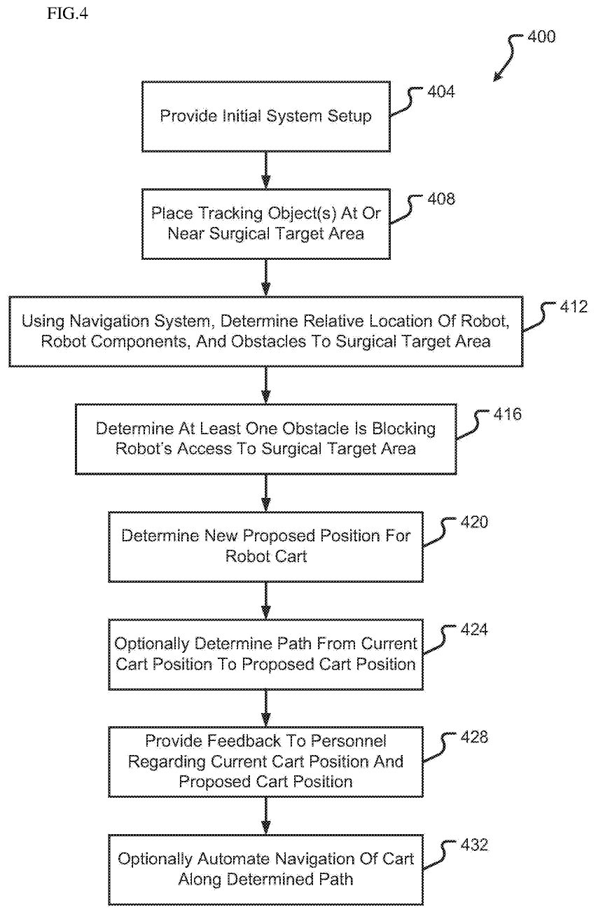

As discussed above, locations of objects (e.g., the table 226, the surgical target area 312, obstacles 320, personnel 324, the robot 114, etc.) within the environment 300 may be determined, at least in part, by the navigation system 118 using image data captured by imaging device(s) 112. Such information may be used to determine whether an initial layout of the environment 300 coincides with a defined layout, whether the initial layout of the environment 300 can be improved to support improved efficiencies in the surgical procedure, whether the layout of the environment 300 has changed such that a new position of the robot 114 is needed or desired, etc. Additional details regarding processes for determining such layouts and suggested improvements for the same will now be described with reference to FIGS. 4-6. In the following description of process flows, it should be appreciated that operations may be performed in a different order than the order shown, or the operations may be performed in different orders or at different times. Certain operations may also be left out of a process flow, or other operations may be added to the process flow. It should also be appreciated that operations from one process flow may be added to other process flows without departing from the scope of the present disclosure.

FIG. 4 illustrates a first example of a process flow 400 in accordance with aspects of the present disclosure. In some examples, process flow 400 may implement aspects of a computing device 102, an imaging device 112, a robot 114, and/or a navigation system 118, described with reference to FIGS. 1-3.

The process flow 400 begins by providing an initial system setup (step 404).

The initial system setup may correspond to an initial layout of objects within the environment 300 and may correspond to an initial operating room layout. In the initial layout, the robot 114 may have a first position relative to the patient 204, the table 126, and to a surgical target area 312.

The flow 400 continues by placing one or more tracking objects 208 at or near the surgical target area (step 408). The one or more tracking objects 208 may be placed on or mounted to the patient 204, the robot 114, an end effector 224 of the robot 114, a robotic arm 216, 220, a surgical instrument, personnel 324, an obstacle 320, or the like. It may be desirable to place the tracking object(s) 208 in a field of view of the imaging device(s) 112 to enable tracking, surgical navigation, robotic movements, etc. In particular, the navigation system 118 may be used to determine a location of the robot 114, components of the robot 114, and/or obstacles 320 relative to a surgical target area 312 (step 412).

As the navigation system 118 tracks location(s) of the objects within the environment 300 (e.g., based on image data), the navigation system 118 may determine that at least one obstacle 320 is blocking the robot's 114 access to the surgical target area 312 (step 416). When an obstacle 320 is identified as blocking the robot's 114 access to the surgical target area 312, either partially or completely, the flow 400 may continue by determining a new proposed cart position 308 for the robot 114 (step 420). The location of the new proposed cart position 308 may correspond to a different position relative to the table 226 that would allow the robot 114 to better access the surgical target area 312. The new proposed cart position 308 may also correspond to a position that is accessible from the current cart position 304 via a safe and acceptable proposed cart path 328 (step 424). If no safe and accessible cart path is available, then the flow 400 may stop as a new proposed cart position 308 may not be available.

After a new proposed cart position 308 is determined, the flow 400 may proceed by providing feedback to personnel 324 regarding the current cart position 304 and the new proposed cart position 308 (step 428). The feedback provided to personnel 324 may include a display of the environment 300 and the layout of objects in the environment 300 (e.g., a map-type display). The feedback provided to personnel 324 may also include indications of whether or not the robot 114 has been moved to a position that coincides with the proposed cart position 308 (e.g., green lights indicating that the robot 114 has been properly moved, red lights indicating that the robot 114 has not been properly moved, etc.). The feedback provided to personnel 324 may also include a depiction of an animation for moving the robot 114 from the current cart position 304 to the proposed cart position 308 (e.g., via the proposed cart path 328). The feedback provided to personnel 324 may also include an indication that the current cart position 304 is the optimal cart position.

The flow 400 may also include an optional step of enabling the robot 114 to autonomously or semi-autonomously move from the current cart position 304 to the proposed cart position 308 via the proposed cart path 328 (step 432). In some embodiments, the movable base 212 may include automated motor control components that autonomously move the robot 114 within the environment. If such autonomous or semi-autonomous movements are enabled, then a controller of the movable base 212 may also be provided with collision avoidance capabilities to ensure that the robot 114 does not impact obstacles 320, personnel 324, or other objects in the environment 300.

Referring now to FIG. 5, details of a second example of a process flow 500 will be described in accordance with aspects of the present disclosure. In some examples, process flow 500 may implement aspects of a computing device 102, an imaging device 112, a robot 114, and/or a navigation system 118, described with reference to FIGS. 1-3.

The flow 500 may begin by determining a current cart position 304 is sub-optimal for enabling the robot 114 to access the surgical target area 312 (step 504). The determination of step 504 may be made in response to patient 204 movement, a change in position of the surgical target area 312, a movement of an obstacle 320, or any other changing condition that might occur during a surgical procedure.

Upon determining that the current cart position 304 is sub-optimal, the flow 500 may continue by determining an improved or optimal cart position, that is different from the current cart position 304 (step 508). The flow 500 may then provide instructions and/or animations via the display 242 for placing the robot 114 at the improved or optimal position (step 512). In particular, personnel 324 may be presented with a display or animation showing the proposed cart path 328. It may also be possible to provide instructions for moving the movable base 212 to a controller of the movable base 212. The instructions may be executed by the controller of the movable base 212 to enable autonomous or semi-autonomous movement of the movable base 212 and robot 114.

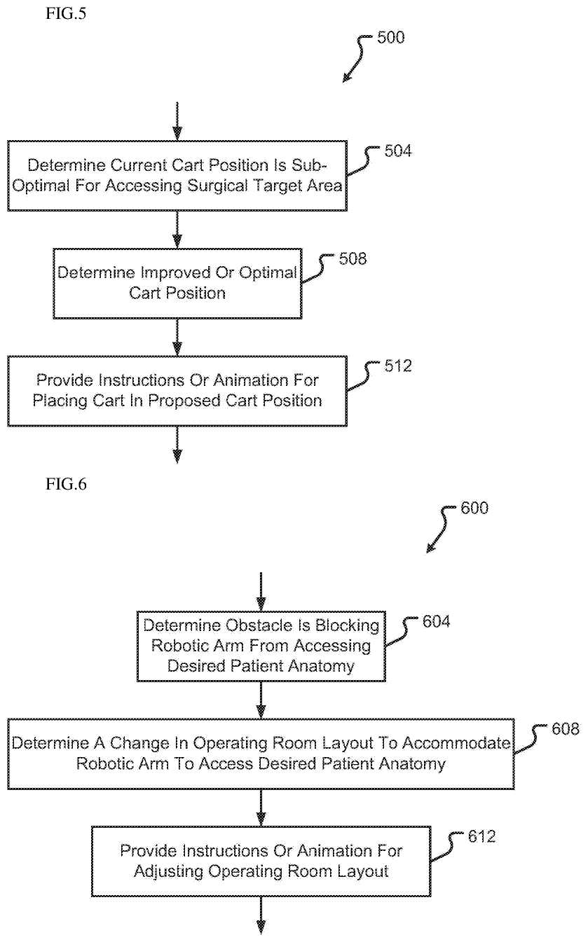

Referring now to FIG. 6, details of a third example of a process flow 600 will be described in accordance with aspects of the present disclosure. In some examples, process flow 600 may implement aspects of a computing device 102, an imaging device 112, a robot 114, and/or a navigation system 118, described with reference to FIGS. 1-3.

The flow 600 may begin by determining an obstacle 320 is blocking a robotic arm 216, 220 from accessing a position or pose, thereby preventing the robot 114 from accessing a desired patient anatomy (step 604). The desired patient anatomy may correspond or be co-located with the surgical target area 312, although the desired patient anatomy does not necessarily need to be co-located with the surgical target area 312.

Upon determining that an obstacle 320 is blocking the robot 114 from a desired position or pose, the flow 600 may continue by determining a change in operating room layout to accommodate the robotic arm 216, 220 better access to the desired patient anatomy (step 608). The flow 600 may then provide instructions and/or animations via the display 242 adjusting the operating room layout (step 612). In particular, changes to the operating room layout may include changing a position of the robot 114, changing a position of the table 226, changing a position of the obstacle 320, or combinations thereof.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C, ” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

1. A system, comprising:
a robot mounted to a movable base, the robot comprising one or more robotic arms;
a processor; and
memory coupled with the processor, the memory comprising data stored thereon that, when processed by the processor, enables the processor to:
receive image data describing a position of the one or more robotic arms relative to a surgical target area;
determine a current position of the robot is sub-optimal for enabling the robot to access the surgical target area; and
provide at least one of instructions and animations for moving the movable base from the current position to a new position.

2. The system of claim 1, wherein the instructions cause the movable base to autonomously move from the current position to the new position.

3. The system of claim 1, wherein the data, when processed by the processor, further enables the processor to:
determine a proposed path from the current position to the new position.

4. The system of claim 3, wherein the proposed path avoids at least one obstacle and remains in a sterile area.

5. The system of claim 1, wherein the image data comprises an image of one or more tracking objects positioned in proximity with the surgical target area.

6. The system of claim 5, wherein the one or more tracking objects are mounted to at least one of an end effector and the one or more robotic arms.

7. The system of claim 5, wherein the one or more tracking objects are mounted to a patient anatomy.

8. The system of claim 5, wherein the one or more tracking objects are mounted to a surgical instrument.

9. The system of claim 1, wherein the image data is obtained from an imaging device and wherein the imaging device comprises at least one of an infrared imaging device, an optical imaging device, and a video camera.

10. The system of claim 1, wherein the instructions are provided for moving the movable base from the current position to an new position and wherein the instructions include an indication of whether or not the movable base is located in the new position.

11. A navigation system, comprising:
an imaging device;
a processor; and
memory coupled with the processor, the memory comprising data stored thereon that, when processed by the processor, enables the processor to:
receive image data from the imaging device, wherein the image data comprises an image of a robotic arm and an image of a surgical target area;
determine a position of the robotic arm;
determine a position of the surgical target area;
determine, based on the position of the robotic arm and the position of the surgical target area, that a cart supporting the robotic arm is sub-optimally placed relative to the surgical target area; and
provide at least one of instructions and animations for moving the cart from a current position to a new position.

12. The navigation system of claim 11, wherein a tracking object is mounted on or held by the robotic arm, wherein the image data includes an image of the tracking object, and wherein the position of the robotic arm is determined by analyzing the image of the tracking object.

13. The navigation system of claim 11, wherein the data, when processed by the processor, further enables the processor to:
determine a proposed path from the current position to the new position; and
display the proposed path via a user interface.

14. The navigation system of claim 11, wherein the data, when processed by the processor, further enables the processor to:
determine an obstacle is precluding the robotic arm from achieving a desired pose to enable an end effector to access the surgical target area; and
determine that the new position enables the end effector to access the surgical target area.

15. The navigation system of claim 11, wherein the imaging device comprises at least one of an infrared imaging device, an optical imaging device, and a video camera.

16. A method comprising:
receiving image data from an imaging device, wherein the image data comprises an image of a robotic arm and an image of a surgical target area;
determining a position of the robotic arm relative to a position of the surgical target area;
determining, based on the position of the robotic arm relative to the position of the surgical target area, that a cart supporting the robotic arm is sub-optimally placed relative to the surgical target area; and
providing at least one of instructions and animations for moving the cart from a current position to a new position.

17. The method of claim 16, further comprising:
outputting an indication when the cart is co-located with the new position.

18. The method of claim 16, further comprising:
determining a path from the current position to the new position.

19. The method of claim 18, further comprising:
causing the cart to move autonomously along the path.

20. The method of claim 18, wherein the path avoids at least one obstacle and remains in a sterile area.

21-35. (canceled)
